SEQUENCE LISTING (23 sequences)

SEQ ID NO: 1 - protein, 29 aa, Homo sapiens
HSQGTFTSDYSKYLDSRRAQDFVQWLMNT

SEQ ID NO: 2 - protein, 29 aa, synthetic construct
  SITE 16: note = X = alpha-aminoisobutyric acid
HSQGTFTSDYSKYLDXARAEEFVKWLEST

SEQ ID NO: 3 - protein, 29 aa, synthetic construct
  SITE 2: note = X = alpha-aminoisobutyric acid
  SITE 16: note = X = alpha-aminoisobutyric acid
HXQGTFISDKSKYLDXRAAQDFVQWLMDT

SEQ ID NO: 4 - protein, 29 aa, synthetic construct
  MOD_RES 3: note = X = acetylated diaminobutyric acid
  SITE 16: note = X = alpha-aminoisobutyric acid
HSXGTFTSDYSKYLDXRRAADFVAWLLDE

SEQ ID NO: 5 - protein, 29 aa, synthetic construct
  SITE 10: note = X = beta-tyrosine
HSQGTFTSDXSKYLDSRRAQDFVQWLMNT

SEQ ID NO: 6 - protein, 29 aa, synthetic construct
  SITE 16: note = X = beta-serine
HSQGTFTSDYSKYLDXRRAQDFVQWLMNT

SEQ ID NO: 7 - protein, 29 aa, synthetic construct
  SITE 24: note = X = beta-glutamine
HSQGTFTSDYSKYLDSRRAQDFVXWLMNT

SEQ ID NO: 8 - protein, 29 aa, synthetic construct
  SITE 21: note = X = beta-aspartic acid
HSQGTFTSDYSKYLDSRRAQXFVQWLMNT

SEQ ID NO: 9 - protein, 29 aa, synthetic construct
  SITE 5: note = X = beta-threonine
HSQGXFTSDYSKYLDSRRAQDFVQWLMNT

SEQ ID NO: 10 - protein, 29 aa, synthetic construct
  SITE 13: note = X = beta-tyrosine
HSQGTFTSDYSKXLDSRRAQDFVQWLMNT

SEQ ID NO: 11 - protein, 29 aa, synthetic construct
  SITE 17: note = X = beta-arginine
HSQGTFTSDYSKYLDSXRAQDFVQWLMNT

SEQ ID NO: 12 - protein, 29 aa, synthetic construct
  SITE 16: note = X = beta-serine
HSQGTFTSDYSKYLDXARAEEFVKWLEST

SEQ ID NO: 13 - protein, 29 aa, synthetic construct
  SITE 2: note = X = beta-serine
  SITE 16: note = X = alpha-aminoisobutyric acid
HXQGTFTSDYSKYLDXARAEEFVKWLEST

SEQ ID NO: 14 - protein, 29 aa, synthetic construct
  SITE 2: note = X = beta-serine
  SITE 16: note = X = beta-serine
HXQGTFTSDYSKYLDXARAEEFVKWLEST

SEQ ID NO: 15 - protein, 29 aa, synthetic construct
  SITE 2: note = X = alpha-aminoisobutyric acid
  SITE 16: note = X = beta-serine
HXQGTFISDKSKYLDXARAEEFVKWLEST

SEQ ID NO: 16 - protein, 29 aa, synthetic construct
  SITE 2: note = X = beta-serine
  SITE 16: note = X = alpha-aminoisobutyric acid
HXQGTFISDKSKYLDXRAAQDFVQWLMDT

SEQ ID NO: 17 - protein, 29 aa, synthetic construct
  SITE 2: note = X = alpha-aminoisobutyric acid
  SITE 16: note = X = beta-serine
HXQGTFISDKSKYLDXRAAQDFVQWLMDT

SEQ ID NO: 18 - protein, 29 aa, synthetic construct
  SITE 2: note = X = beta-serine
  SITE 16: note = X = beta-serine
HXQGTFISDKSKYLDXRAAQDFVQWLMDT

SEQ ID NO: 19 - protein, 29 aa, synthetic construct
  SITE 2: note = X = alpha-aminoisobutyric acid
  SITE 16: note = X = alpha-aminoisobutyric acid
  SITE 24: note = X = beta-glutamine
HXQGTFISDKSKYLDXRAAQDFVXWLMDT

SEQ ID NO: 20 - protein, 29 aa, synthetic construct
  SITE 3: note = X = beta-glutamine
  SITE 16: note = X = alpha-aminoisobutyric acid
  SITE 24: note = X = beta-glutamine
HSXGTFTSDYSKYLDXRRAADFVXWLLDE

SEQ ID NO: 21 - protein, 29 aa, synthetic construct
  SITE 3: note = X = beta-glutamine
  SITE 16: note = X = alpha-aminoisobutyric acid
HSXGTFTSDYSKYLDXRRAADFVAWLLDE

SEQ ID NO: 22 - protein, 29 aa, synthetic construct
  MOD_RES 3: note = X = acetylated diaminobutyric acid
  SITE 16: note = X = beta-serine
  SITE 24: note = X = beta-glutamine
HSXGTFTSDYSKYLDXRRAADFVXWLLDE

SEQ ID NO: 23 - protein, 29 aa, synthetic construct
  SITE 3: note = X = beta-glutamine
  SITE 16: note = X = beta-serine
HSXGTFTSDYSKYLDXRRAADFVAWLLDE

(19) United States
(12) Patent Application Publication
(10) Pub. No.: US 2026/0274915 A1
(43) Pub. Date: Sep. 17, 2026

(54) Glucagon Analogs Comprising Beta-Amino Acids, Their Pharmaceutically Acceptable Salts, and Therapeutic Applications

(71) Applicants: Ling XUE, Clinton, MA (US); Guibai LIANG, SCOTCH PLAINS, NJ (US)

(72) Inventors: Ling XUE, Clinton, MA (US); Guibai LIANG, SCOTCH PLAINS, NJ (US)

(21) Appl. No.: 19/079,315

(22) Filed: Mar. 13, 2025

(51) Int. Cl. C07K 14/605; A61K 38/26; A61K 45/06

(52) U.S. Cl. CPC C07K 14/605; A61K 38/26; A61K 45/06

(57) ABSTRACT

The present invention discloses a glucagon analogue comprising β-amino acid substitutions or a pharmaceutically acceptable salt thereof, along with its therapeutic applications. By incorporating β-amino acids at designated positions within the glucagon sequence, the disclosed analogues exhibit glucagon receptor (GCGR) agonist activity comparable or superior to native glucagon in in vitro and in vivo assays. Furthermore, these structural modifications confer enhanced plasma stability and resistance to hepatic microsomal degradation, resulting in improved pharmacokinetic profiles and drug-like properties. Collectively, these attributes position the analogues as viable candidates for further development into therapeutic interventions in metabolic disorders such as diabetes, obesity, and hyperglycemia.

### SEQUENCE LISTING

The present invention contains a Sequence Listing submitted in XML format and is incorporated herein by reference in its entirety for all purposes. Said XML is named “LC25210002-US.20250313.Sequence Listing.xml”, was last modified on Mar. 12, 2025, and is 34,892 KB in size. A substitute Sequence Listing is filed electronically herewith, the contents of which are incorporated by reference in their entirety. The electronic file was created on Apr. 8, 2025, is identical, 35,521 bytes in size, and titled 19079315.SEQ002.xml.

### TECHNICAL FIELD

The present invention pertains to the field of biopharmaceuticals, specifically to glucagon analogs comprising β-amino acids and their use in the treatment of metabolic disorders.

### TECHNICAL BACKGROUND

Proglucagon, a 158-amino acid precursor polypeptide, undergoes tissue-specific processing to yield functionally distinct hormones and peptides, including glucagon (Glu), glucagon-like peptide-1 (GLP-1), glucagon-like peptide-2 (GLP-2), and oxyntomodulin (OXM). These derivatives regulate critical physiological processes such as glycemic homeostasis, insulin secretion, gastric motility, intestinal proliferation, and appetite modulation.

Glucagon binds to hepatic glucagon receptors (GCGR), stimulating glycogenolysis to release stored glucose and maintain blood glucose levels. During prolonged fasting, glucagon further activates gluconeogenesis to synthesize glucose, thereby preventing hypoglycemia. Structurally, glucagon corresponds to residues 53-81 of proglucagon, comprising the sequence: His-Ser-Gln-Gly-Thr-Phe-Thr-Ser-Asp-Tyr-Ser-Lys-Tyr-Leu-Asp-Ser-Arg-Arg-Ala-Gln-Asp-Phe-Val-Gln-Trp-Leu-Met-Asn-Thr (SEQ ID NO: 1).

Numerous glucagon analogs have been engineered via amino acid substitution or modification to mimic native glucagon's receptor activation. These analogs function as GCGR agonists, enhancing metabolic rate, oxygen consumption, and energy expenditure while delaying gastric emptying and suppressing appetite—properties advantageous for weight management and glycemic control. However, natural glucagon and existing analogs face limitations such as enzymatic instability in vivo and suboptimal pharmacokinetics (e.g., plasma half-life <10 minutes), hindering their therapeutic utility for conditions such as obesity and impaired glucose tolerance.

### SUMMARY OF THE INVENTION

The present invention addresses these limitations by providing a glucagon analog exhibiting GCGR agonist activity with enhanced stability, specificity, and potency compared to native glucagon and prior analogs. These improvements optimize therapeutic efficacy for metabolic disorders.

### Technical Solution

The invention provides a glucagon analog or pharmaceutically acceptable salt thereof, comprising an amino acid sequence with β-amino acids substituted at one or more positions selected from 2, 3, 5, 10, 13, 16, 17, 21, and 24 relative to SEQ ID NO. 1.

As recognized in the field, β-amino acids structurally resemble naturally occurring α-amino acids but feature an amino group bonded to the β-carbon (C3) instead of the α-carbon (C2).

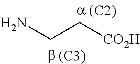

### 1. Glucagon Analogues or Pharmaceutically Acceptable Salts Thereof

The invention provides a glucagon analog or pharmaceutically acceptable salt thereof, comprising an amino acid sequence derived from SEQ ID NO. 1, wherein one or more α-amino acid residues at positions 2, 3, 5, 10, 13, 16, 17, 21, or 24 are substituted with β-amino acid counterparts.

Preferably, the β-amino acid is represented by Formula (I):

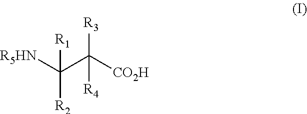

In Formula (I), substituents R1-R5 may independently be hydrogen or substituent groups, with optional cyclic structures formed between substituents and adjacent carbons.

Preferably, R1, R2, R3, R4, and R5 are independently selected from hydrogen or substituent groups, which may be identical or distinct; and/or, any two of R1, R2, R3, R4, or R5, together with the α-carbon (C2) and/or β-carbon (C3) to which they are bonded, may form a cyclic structure. Preferably, the β-amino acid is represented by Formula (II):

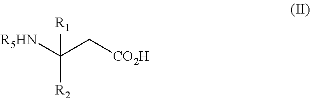

In Formula (II), substituents R1, R2, and R5 are independently selected from hydrogen or substituent groups, which may be identical or distinct; and/or, any two of R1, R2, or R5, together with the β-carbon (C3) to which they are bonded, may form a cyclic structure.

Formula II further specifies preferred configurations where R5 is hydrogen, and cyclic structures may involve R1 or R2. Preferably, R5 may be hydrogen (H). Preferably, at least one of R1 or R2 may be hydrogen (H).

Preferably, the β-amino acids may retain the side chain moieties of their α-amino acid counterparts, differing in amino group positioning and the insertion of methylene unit in the backbone. The β-amino acids may exist in the amino acid sequence as shown below:

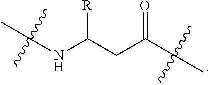

Substitutions may occur at 2-9 designated sites, numbered per SEQ ID NO: 1. Exemplary substitutions include β-serine (βS), β-glutamine (βQ), β-threonine (βT), β-tyrosine (βY), β-arginine (βR), and β-aspartic acid (βD).

### PREFERRED EMBODIMENTS

### 1. β-Amino Acid Substitutions:

Single or dual substitutions at positions 2, 3, 5, 10, 13, 16, 17, 21, or 24 (e.g., βY at position 10, βS at 16, βQ at 24).
Specific combinations such as βS at positions 2 and 16, or βQ at positions 3 and 24.

For example, the substitutions comprise:
(1) βY at position 10;
(2) βS at position 16,
(3) βQ at position 24;
(4) βD at position 21;
(5) βT at position 5;
(6) βY at position 13;
(7) βR at position 17;
(8) βS at position 2;
(9) βS at positions 2 and 16;
(10) βQ at positions 3 and 24;
(11) βQ at position 3;
(12) βS at position 16 and βQ at position 24; or
(13) βQ at position 3 and βS at position 16.

### 2. Additional Amino Acid Modifications:

Substitutions at positions 7, 10, 17, 18, 20, 21, 24, 27, 28, and/or 29, including T7I, Y10K, R17A, R18A, Q20A, Q20E, D21E, Q24K, Q24A, M27E, M27L, N28S, N28D, and T29E.
Preferred combinations:
   R17A, Q20B, D21E, Q24K, M27E, N28S
   T7I, Y10K, R17A, Q20E, D21E, Q24K, M27E, N28S
   T7I, Y10K, R18A, N28D
   Q20A, M27L, N28D, T29E,
   Q20A, Q24A, M27L, N28D, T29E

### 3. Non-Natural Amino Acids:

Incorporation of acetyl-2,4-diaminobutyric acid (DAB (Ac)) or α-aminoisobutyric acid (Aib) at positions 2, 3, or 16.
Preferred combinations:
   Aib at position 16
   Aib at position 2
   DAB (Ac) at position 3
   Aib at positions 2 and 16

### CONCLUSION

The disclosed glucagon analogs integrate β-amino acids, targeted substitutions, and non-natural residues to confer resistance to proteolytic degradation, prolonged half-life, and enhanced receptor specificity. These innovations address unmet clinical needs in metabolic disease therapeutics, offering improved efficacy for conditions such as obesity and diabetes mellitus. Exemplary amino acid sequences comprising the glucagon analogues or pharmaceutically acceptable salts thereof disclosed in the present invention are enumerated in Table 1:

TABLE 1
Amino acid sequences of polypeptides

| | | | | Additional | Additional amino |
|---|---|---|---|---|---|
| | | Amino acid | | unnatural | acid substitutions |
| Polypeptide | | sequence | β-amino | amino | relative to SEQ |
| number | Amino acid sequence | number | acid(s) | acid(s) | ID NO. 1 |
| glucagon | HSQGTFTSDYSKYLDSRRAQ | SEQ ID NO. 1 | / | / | / |
| | DFVQWLMNT | | | | |
| P001 | HSQGTFTSDXSKYLDSRRAQ | SEQ ID NO. 5 | Y10X = βY | | |
| | DFVQWLMNT | | | | |
| P002 | HSQGTFTSDYSKYLDXRRAQ | SEQ ID NO. 6 | S16X = βS | | |
| | DFVQWLMNT | | | | |
| P003 | HSQGTFTSDYSKYLDSRRAQ | SEQ ID NO. 7 | Q24X = βQ | | |
| | DFVXWLMNT | | | | |
| P004 | HSQGTFTSDYSKYLDSRRAQ | SEQ ID NO. 8 | D21X = βD | | |
| | XFVQWLMNT | | | | |
| P005 | HSQGXFTSDYSKYLDSRRAQ | SEQ ID NO. 9 | T5X = βT | | |
| | DFVQWLMNT | | | | |
| P006 | HSQGTFTSDYSKXLDSRRAQ | SEQ ID NO. 10 | Y13X = βY | | |
| | DFVQWLMNT | | | | |
| P007 | HSQGTFTSDYSKYLDSXRAQ | SEQ ID NO. 11 | R17X = βR | | |
| | DFVQWLMNT | | | | |
| P008 | HSQGTFTSDYSKYLDXARAE | SEQ ID NO. 12 | S16X = βS | | R17A; Q20E; |
| | EFVKWLEST | | | | D21E; Q24K; |
| | | | | | M27E; N28S |
| P009 | HXQGTFTSDYSKYLDXARAE | SEQ ID NO. 13 | S2X = βS | S16X = Aib | R17A; Q20E; |
| | EFVKWLEST | | | | D21E; Q24K; |
| | | | | | M27E; N28S |
| P010 | HXQGTFTSDYSKYLDXARAB | SEQ ID NO. 14 | S2X = βS; | | R17A; Q20E; |
| | EFVKWLEST | | S16X = βS | | D21E; Q24K; |
| | | | | | M27E; N28S |
| P011 | HXQGTFISDKSKYLDXARAEE | SEQ ID NO. 15 | S16X = βS | S2X = Aib | T7I; Y10K; R17A; |
| | FVKWLEST | | | | Q20E; D21E; |
| | | | | | Q24K; M27E; N28S |
| P012 | HXQGTFISDKSKYLDXRAAQ | SEQ ID NO. 16 | S2X = βS | S16X = Aib | T7I; Y10K; R18A; |
| | DFVQWLMDT | | | | N28D |
| P013 | HXQGTFISDKSKYLDXRAAQ | SEQ ID NO. 17 | S16X = βS | S2X = Aib | T7I; Y10K; R18A; |
| | DFVQWLMDT | | | | N28D |
| P014 | HXQGTFISDKSKYLDXRAAQ | SEQ ID NO. 18 | S2X = βS; | | T7I; Y10K; R18A; |
| | DFVQWLMDT | | S16X = βS | | N28D |
| P015 | HXQGTFISDKSKYLDXRAAQ | SEQ ID NO. 19 | Q24X = βQ | S2X, S16X | T7I; Y10K; R18A; |
| | DFVXWLMDT | | | = Aib | N28D |
| P016 | HSXGTFTSDYSKYLDXRRAA | SEQ ID NO. 20 | Q3X = βQ; | S16X = Aib | Q20A; M27L; |
| | DFVXWLLDE | | Q24X = βQ | | N28D; T29E |
| P017 | HSXGTFTSDYSKYLDXRRAA | SEQ ID NO. 21 | Q3X = βQ | S16X = Aib | Q20A; Q24A; |
| | DFVAWLLDE | | | | M27L; N28D; T29E |
| P018 | HSXGTFTSDYSKYLDXRRAA | SEQ ID NO. 22 | S16X = βS; | Q3X = | Q20A; M27L; |
| | DFVXWLLDE | | Q24X = βQ | Dab(Ac) | N28D; T29E |
| P019 | HSXGTFTSDYSKYLDXRRAA | SEQ ID NO. 23 | Q3X = βQ; | | Q20A; Q24A; M27L; |
| | DFVAWLLDE | | S16X = βS | | N28D; T29E |

In specific embodiments of the present invention, the disclosed glucagon analogue comprises a polypeptide, including, but not limited to, those designated as P001 to P019 in Table 1. The amino acid sequences of said polypeptides correspond to those exemplified in SEQ ID Nos. 5-23.

### Pharmaceutically Acceptable Salts of Glucagon Analogues

The present invention further encompasses pharmaceutically acceptable salts of the disclosed glucagon analogues, wherein the glucagon analogue is as defined herein.

The pharmaceutically acceptable salts may include inorganic acid salts, organic acid salts, inorganic base salts, or organic base salts. Non-limiting examples of inorganic acid salts comprise hydrochloride, sulfate, phosphate, hydrobromide, and hydroiodide. Organic acid salts may include, but are not limited to, formate, acetate, propionate, benzoate, maleate, methanesulfonate, fumarate, trichloroacetate, trifluoroacetate, and citrate. Inorganic base salts are exemplified by sodium, potassium, lithium, calcium, magnesium, ammonium, aluminum, zinc, copper, or manganese salts. Organic base salts include those derived from organic bases such as methylamine, dimethylamine, trimethylamine, piperidine, morpholine, or tri-(2-hydroxyethyl)amine.

### 2. Pharmaceutical Compositions

In another aspect, the invention provides a pharmaceutical composition comprising a glucagon analogue or a pharmaceutically acceptable salt thereof as described herein, optionally formulated with one or more pharmaceutically acceptable carriers, excipients, or adjuvants.

The composition may be formulated into dosage forms selected from the group consisting of tablets, capsules, syrups, tinctures, inhalants, sprays, injections, films, patches, powders, granules, emulsions, suppositories, or combination preparations.

Optionally, the composition may further comprise adjunctive agents compatible with the glucagon analogues or salts thereof. Such agents may include natural glucagon or synthetic glucagon analogues. The synthetic analogues may act as agonists of G protein-coupled receptors (GPCRs), including but not limited to GCGR, GLP1R, and GIPR agonists.

### 3. Therapeutic Applications

The invention further discloses the use of the glucagon analogue, its pharmaceutically acceptable salt, or the pharmaceutical composition in the manufacture of a medicament. Said medicament functions as a glucagon receptor agonist and is suitable for the treatment or prevention of disorders associated with insulin secretion, glucagon secretion, or appetite regulation. Non-limiting indications include type 1 diabetes, type 2 diabetes, hyperglycemia, hypoglycemia, glucagon deficiency, overweight, obesity, and bulimia.

### 4. Treatment Methods

A method for treating or preventing disorders related to insulin secretion, glucagon secretion, or appetite regulation is also provided, comprising administering to a subject in need thereof a therapeutically or prophylactically effective amount of the glucagon analogue, its pharmaceutically acceptable salt, or the pharmaceutical composition. The disorders include, but are not limited to, type 1 diabetes, type 2 diabetes, hyperglycemia, hypoglycemia, glucagon deficiency, overweight, obesity, and bulimia.

As used herein, the term “subject” refers to a mammal, preferably a primate (e.g., human) or rodent.

### 5. Structural Novelty and Advantages

The invention introduces novel glucagon analogues incorporating β-amino acids. Relative to natural glucagon, the analogues feature substitution of α-amino acids with β-amino acids at specific positions within the amino acid sequence.

Experimental data confirm that such substitutions yield modified glucagon analogues with retained or enhanced glucagon receptor agonist activity. These analogues exhibit comparable or superior receptor activation potency, alongside markedly improved plasma stability and hepatic microsomal stability. Consequently, the analogues demonstrate enhanced drug-like properties, rendering them suitable for therapeutic use in metabolic disorders such as obesity, diabetes, and related conditions. Furthermore, the analogues exhibit superior specificity for the glucagon receptor (GCGR) over GLP1R and GIPR, minimizing off-target effects.

### DETAILED DESCRIPTION

The present invention is herein described through illustrative embodiments. It is to be understood by those skilled in the art that the following examples are provided solely to elucidate the invention and are not intended to limit its scope in any manner.

In the embodiments below, unless expressly stated otherwise, all experimental protocols adhere to well-established methodologies. Reagents, materials, and related components employed herein are commercially sourced unless specified.

### Glucagon and Exemplary Glucagon Analogues:

1. Glucagon:
(SEQ ID NO. 1)
HSQGTFTSDYSKYLDSRRAQDFVQWLMNT.
2. Dasiglucagon:
(SEQ ID NO. 2)
HSQGTFTSDYSKYLDX₁₆ARAEEFVKWLEST.
wherein X₁₆ denotes α-aminoisobutyric
acid (Aib)
3. TUB288:
(SEQ ID NO. 3)
HX2QGTFISDKSKYLDX₁₆RAAQDFVQWLMDT.
wherein X₂ and X₁₆ each
denotes α-aminoisobutyric acid (Aib)
4. IUB76:
(SEQ ID NO. 4)
HSX₃GTFTSDYSKYLDX₁₆RRAADFVAWLLDE,
wherein X₃ represents acetylated
diaminobutyric acid (Dab(Ac)), and X₁₆ denotes
α-aminoisobutyric acid (Aib)

### Example 1: Synthesis of 6-Amino Acid-Containing Polypeptides

The β-amino acid-containing polypeptides enumerated in Table 1 were synthesized via Fmoc-based solid-phase peptide synthesis (SPPS) employing an automated peptide synthesizer.

Reactions were conducted using CTC resin (triphenylmethyl chloride resin; loading: 0.51 mmol/g) at a synthesis scale of 0.2 mmol.

### Resin Activation and Initial Coupling

CTC resin (1 g) was suspended in dichloromethane (DCM, 10 mL) and agitated at 25° C. (400 rpm, 30 minutes) to activate the resin. The first amino acid (4.0 equivalents relative to resin loading) was coupled to the activated CTC resin under Fmoc protection. Adjustments to amino acid stoichiometry may be implemented to modulate resin substitution levels as required.

### Amino Acid Preparation and Coupling

Fmoc-protected amino acid (1 g) was dissolved in DCM (10 mL), with incremental additions of dimethylformamide (DMF) as needed to achieve complete solubilization. The amino acid solution was combined with the resin suspension, followed by diisopropylcarbodiimide (DIC, 4.0 equivalents). The mixture was agitated at 25° C. (400 rpm, 1 hour). Residual trityl groups were quenched with HPLC-grade methanol (0.8 mL/g resin) under agitation (25° C., 15 minutes). The resin was then sequentially washed with DCM (3×10 mL/g resin) and DMF (2×10 mL/g resin) via vacuum filtration.

### Fmoc Deprotection

Fmoc deprotection was performed using 20% (v/v) piperidine in DMF under agitation (400 rpm, 10 minutes), followed by resin washing and immediate utilization in subsequent coupling cycles.

### Sequential Amino Acid Coupling

Natural amino acids (4.0 equivalents, side-chain protected) were coupled in DMF (2.0 mL per 150 mg resin) using Oxyma (4.0 equivalents, CAS: 3849-21-6) and DIC (8.0 equivalents, CAS: 693-13-0) at 50° C. (400 rpm, 45 minutes). Non-natural β-amino acids (4.0 equivalents, appropriately protected) underwent analogous coupling conditions with extended reaction duration (1 hour).

### Post-Coupling Processing

Post-coupling, the resin was exhaustively washed with DMF (4×3 mL/wash) prior to subsequent synthesis cycles. Iterative Fmoc removal and coupling procedures were repeated until full sequence assembly was achieved. Terminal Fmoc deprotection was conducted as previously described.

### Global Deprotection and Cleavage

Global deprotection and resin cleavage were performed using a cocktail of TFA:H₂O:thioanisole:DODT (CAS: 14970-87-7; 37:1:1:1 v/v) under nitrogen atmosphere at 40° C. (2 hours). The cleaved peptide was precipitated in cold diethyl ether (50 mL), incubated at −20° C. (30 minutes), and pelleted via centrifugation (4400 g, 5 minutes).

### Purification and Characterization

The crude peptide was purified by reversed-phase HPLC (conditions detailed in Table 2). Purified fractions were characterized by MALDI-TOF mass spectrometry (Bruker UltraFlexXtreme II) in reflection mode (positive/negative ion detection). Lyophilization of validated fractions yielded the target polypeptide as a solid.

TABLE 2
Purification of Polypeptides

| Polypeptide | |
|---|---|
| number | Purification conditions |
| P001 | Mobile phase A: water (0.1% TFA), mobile phase B: acetonitrile; |
| | Chromatographic column: Welch XT-C18, 30 * 150 mm, 10 μm; Gradient elution: |
| | 18% B to 39% B, 20 minutes, flow rate: 35 mL/min; Wavelength: 220 nm. |
| P002 | Mobile phase A: water (0.1% TFA), mobile phase B: acetonitrile; |
| | Chromatographic column: Welch XT-C18, 30 * 150 mm, 10 μm; Gradient elution: |
| | 19% B to 39% B, 20 minutes, flow rate: 35 mL/min; Wavelength: 220 nm. |
| P003 | Mobile phase A: water (0.1% TFA), mobile phase B: acetonitrile; |
| | Chromatographic column: Welch XT-C18, 30 * 150 mm, 10 μm; Gradient elution: |
| | 16% B to 37% B, 20 minutes, flow rate: 35 mL/min; Wavelength: 220 nm. |
| P004 | Mobile phase A: water (0.1% TFA), mobile phase B: acetonitrile; |
| | Chromatographic column: Welch XT-C18, 30 * 150 mm, 10 μm; Gradient elution: |
| | 16% B to 37% B, 20 minutes, flow rate: 35 mL/min; Wavelength: 220 nm. |
| P005 | Mobile phase A: water (0.1% TFA), mobile phase B: acetonitrile; |
| | Chromatographic column: Welch XT-C18, 30 * 150 mm, 10 μm; Gradient elution: |
| | 22% B to 38% B, 20 minutes, flow rate: 35 mL/min; Wavelength: 220 nm. |
| P006 | Mobile phase A: water (0.1% TFA), mobile phase B: acetonitrile; |
| | Chromatographic column: Welch XT-C18, 30 * 150 mm, 10 μm; Gradient elution: |
| | 18% B to 38% B, 20 minutes, flow rate: 35 mL/min; Wavelength: 220 nm. |
| P007 | Mobile phase A: water (0.1% TFA), mobile phase B: acetonitrile; |
| | Chromatographic column: Welch XT-C18, 30 * 150 mm, 10 μm; Gradient elution: |
| | 18% B to 38% B, 20 minutes, flow rate: 35 mL/min; Wavelength: 220 nm. |
| P008 | Mobile phase A: water (0.1% TFA), mobile phase B: acetonitrile; |
| | Chromatographic column: Welch XT-C18, 30 * 150 mm, 10 μm; Gradient elution: |
| | 18% B to 38% B, 20 minutes, flow rate: 35 mL/min; Wavelength: 220 nm. |
| P009 | Mobile phase A: water (0.1% TFA), mobile phase B: acetonitrile; |
| | Chromatographic column: Welch XT-C18, 30 * 150 mm, 10 μm; Gradient elution: |
| | 18% B to 45% B, 20 minutes, flow rate: 35 mL/min; Wavelength: 220 nm. |
| P010 | Mobile phase A: water (0.1% TFA), mobile phase B: acetonitrile; |
| | Chromatographic column: Welch XT-C18, 30 * 150 mm, 10 μm; Gradient elution: |
| | 18% B to 38% B, 20 minutes, flow rate: 35 mL/min; Wavelength: 220 nm. |
| P011 | Mobile phase A: water (0.1% TFA), mobile phase B: acetonitrile; |
| | Chromatographic column: Welch XT-C18, 30 * 150 mm, 10 μm; Gradient elution: |
| | 18% B to 36% B, 20 minutes, flow rate: 35 mL/min; Wavelength: 220 nm. |
| P012 | Mobile phase A: water (0.1% TFA), mobile phase B: acetonitrile; |
| | Chromatographic column: Welch XT-C18, 30 * 150 mm, 10 μm; Gradient elution: |
| | 22% B to 44% B, 20 minutes, flow rate: 35 mL/min; Wavelength: 220 nm. |
| P013 | Mobile phase A: water (0.1% TFA), mobile phase B: acetonitrile; |
| | Column: Welch XT-C18, 30*150 mm, 10 μm; Gradient elution: 20% B |
| | To 43% B, 20 minutes, flow rate: 35 ml/min; Wavelength: 220 nm. |
| P014 | Mobile phase A: water (0.1% TFA), mobile phase B: acetonitrile; |
| | Chromatographic column: Welch XT-C18, 30 * 150 mm, 10 μm; Gradient elution: |
| | 22% B to 42% b for 20 minutes, flow rate: 35 mL/min; Wavelength: 220 nm. |
| P015 | Mobile phase A: water (0.1% TFA), mobile phase B: acetonitrile; |
| | Chromatographic column: Welch XT-C18, 30 * 150 mm, 10 μm; Gradient elution: |
| | 22% B to 46% b for 20 minutes, flow rate: 35 mL/min; Wavelength: 220 nm. |
| P016 | Mobile phase A: water (0.1% TFA), mobile phase B: acetonitrile; |
| | Chromatographic column: Welch XT-C18, 30 * 150 mm, 10 μm; Gradient elution: |
| | 17% B to 46% B, 20 minutes, flow rate: 35 mL/min; Wavelength: 220 nm. |
| P017 | Mobile phase A: water (0.1% TFA), mobile phase B: acetonitrile; |
| | Chromatographic column: Welch XT-C18, 30 * 150 mm, 10 μm; Gradient elution: |
| | 22% B to 47% B, 20 minutes, flow rate: 35 mL/min; Wavelength: 220 nm. |
| P018 | Mobile phase A: water (0.1% TFA), mobile phase B: acetonitrile; |
| | Chromatographic column: Welch XT-C18, 30 * 150 mm, 10 μm; Gradient elution: |
| | 17% B to 40% B, 20 minutes, flow rate: 35 mL/min; Wavelength: 220 nm. |
| P019 | Mobile phase A: water (0.1% TFA), mobile phase B: acetonitrile; |
| | Column Welch XT-C18, 30 * 150 mm, 10 μm; Gradient elution: 22% B to 38% B, |
| | 20 minues, flow rate: 35 mL/min; Wavelength: 220 nm. |

### Example 2: Characterization of β-Amino Acid-Containing Polypeptides as Agonists for Class B GPCRs (GCGR, GLP1R, GIPR)

The in vitro agonist potency of the β-amino acid-containing polypeptides disclosed herein was evaluated against the glucagon receptor (GCGR), glucagon-like peptide-1 receptor (GLP1R), and gastric inhibitory peptide receptor (GIPR) using a homogeneous time-resolved fluorescence (HTRF) assay to quantify intracellular cAMP levels.

### 1. Reagents and Cell Lines

Control Polypeptides:
   GLP1 (7-37): Sourced from MedChemExpress (MCE).
   GIP (1-42): Procured from Tocris Bioscience.
   Glucagon hydrochloride: Obtained from Selleck Chemicals.
Assay Buffer: Hanks' balanced salt solution (HBSS) supplemented with 20 mM HEPES, 0.1% (w/v) bovine serum albumin (βSA), and 0.5 mM 3-isobutyl-1-methylxanthine (IBMX) (pH 7.4).
Recombinant Cell Lines:
   Flp-In-293-GLP1R, Flp-In-293-GIPR, and Flp-In-CHO-GCGR cells were generated using the Flp-In™ system (Thermo Fisher Scientific, Loughborough, UK) for stable overexpression of GLP1R, GIPR, and GCGR, respectively. Cells were cryopreserved in liquid nitrogen and thawed immediately prior to experimentation.

### 2. Experimental Protocol

### Polypeptide Dilution:

Test and control polypeptides were serially diluted in assay buffer to generate 10 concentration gradients.

### Cell Preparation:

Stably transfected Flp-In-CHO-GCGR cells were resuspended in assay buffer and co-incubated with polypeptide solutions in assay plates.

### CAMP Quantification:

Following a 30-minute incubation at 37° C., intracellular cAMP levels were measured using the CAMP Dynamic 2 HTRF assay kit (Cisbio, Codolet, France) per manufacturer guidelines. Plates were read after 1 hour using an En Vision multimode plate reader (PerkinElmer, Waltham, MA, USA).

### Cross-Receptor Profiling:

Agonist activity against GLP1R and GIPR was similarly assessed using Flp-In-293-GLP1R and Flp-In-293-GIPR cells, respectively, at a test polypeptide concentration of 1 nM.

### 3. Data Analysis

Dose-response curves were generated via nonlinear regression analysis (GraphPad Prism 9.0, San Diego, CA, USA) to determine half-maximal effective concentration (EC) values. Results are summarized in Tables 3 and 4.

TABLE 3
Excitative effect of polypeptide on glucagon receptor

| Test article | EC50 (nM) | Relative effectiveness (%) |
|---|---|---|
| glucagon | 0.00205 | 100 |
| P001 | 0.00883 | 24.56 |
| P002 | 0.00469 | 41.20 |
| P003 | 0.00188 | 102.71 |
| P004 | 0.00412 | 46.82 |
| P005 | 0.06278 | 3.45 |
| P006 | 0.00786 | 27.58 |
| P007 | 0.00998 | 21.72 |
| P008 | 0.05780 | 3.75 |
| P009 | 0.23228 | 0.93 |
| P010 | >1 | <0.19 |
| P011 | >1 | <0.19 |
| P012 | >1 | <0.22 |
| P013 | 0.18920 | 1.02 |
| P014 | >1 | <0.19 |
| P015 | 0.08390 | 2.30 |
| P016 | 0.02455 | 8.83 |
| P017 | 0.03255 | 6.66 |
| P018 | 0.00133 | 163.29 |
| P019 | 0.10146 | 2.14 |
| Dasiglucagon | 0.00670 | 14.61 |
| IUB288 | 0.23225 | 1.21 |
| IUB76 | 0.00320 | 88.25 |

As shown in Table 3, the β-amino acid-containing polypeptides disclosed herein exhibit potent agonist activity on the glucagon receptor (GCGR), inducing a significant elevation in glucose production under in vitro assay conditions. The observed efficacy underscores their functional equivalence or superiority to native glucagon, validating their therapeutic potential for metabolic disorders requiring glucagon receptor modulation.

TABLE 4
Excitative effect of polypeptides on glucagon-like peptide 1 receptor
(GLP-1R) and gastric inhibitory peptide receptor (GIPR)

| Test article | GLP1R | GIPR |
|---|---|---|
| GLP1 | 100% | |
| GIP | | 100% |
| P003 | 79% | −8% |
| P008 | 13% | −4% |
| P015 | 9% | −6% |
| P016 | 9% | −7% |
| P018 | 27% | 3% |

As shown in Table 4, the β-amino acid-containing polypeptides disclosed herein demonstrate marked selectivity for the glucagon receptor (GCGR) relative to GLP1R and GIPR, exhibiting ≥10-fold higher binding affinity in comparative agonist assays. This receptor-specific activity profile minimizes off-target interactions while preserving therapeutic efficacy in glucose homeostasis modulation.

### Example 3: Characterization of Plasma Stability and Hepatic Microsomal Metabolic Stability of β-Amino Acid-Containing Polypeptides

The plasma stability and hepatic microsomal metabolic stability profiles of the β-amino acid-containing polypeptides disclosed herein were evaluated in both human and murine biological matrices to assess their pharmacokinetic resilience.

### 1. Plasma Stability Assay

### Experimental Procedure:

Working solutions (1 mM) of the β-amino acid-containing polypeptides and control polypeptides were prepared in dimethyl sulfoxide (DMSO). For each compound, 4 μL of the working solution was introduced into 796 μL of pre-equilibrated human or mouse plasma, yielding a final peptide concentration of 5 μM. Aliquots (50 μL) of the plasma-peptide mixture were dispensed into fresh reaction tubes and incubated in a shaking water bath at 37° C. (60 rpm) for predetermined intervals: min (T15), 30 min (T30), 60 min (T60), and 120 min (T120). Reaction termination was achieved at each time point by addition of 50 μL of 1N HCl and 300 μL of a pre-chilled quenching solution (acetonitrile containing 3% formic acid, 500 nM labetalol, and 2 μM tolfenamic acid as internal standards). The TO control was prepared by immediate quenching of fresh plasma with HCl and the quenching solution prior to incubation.

Post-quenching, samples were vortexed for 5 min and centrifuged at 4° C. (3220 g, 30 min) to pelletize proteins. Subsequently, 100 μL of the clarified supernatant was diluted with an equal volume of ultrapure water in a 96-well plate for subsequent liquid chromatography-tandem mass spectrometry (LC-MS/MS) analysis.

### Data Analysis:

The peak area ratio for each analyte was quantified from extracted ion chromatograms. The residual peptide percentage at each time point was computed using the formula:

$$Remaining percentage at t min ( \% ) = ( \frac{Peak area at t min}{Peak area at T 0} ) \times 100$$

Here, the remaining percentage at t min corresponds to the ratio of the target or control polypeptide peak area at time t relative to the initial peak area (70). Half-life (t½) values were extrapolated from the resulting degradation kinetics.

### 2. Hepatic Microsomal Metabolic Stability Assessment

### Experimental Protocol:

Stock solutions of the test compounds and reference controls were prepared in accordance with the parameters specified in Table 5.

TABLE 5
Preparation of Stock Solutions

| | Storage | | | | Final | |
|---|---|---|---|---|---|---|
| Reagent | concentration | | Volume | | concentration | |
| Phosphate buffer | 200 | mM | 200 | μL | 100 | mM |
| Ultrapure H₂O | — | | 66 | μL | — | |
| MgCl₂ solution | 50 | mM | 40 | μL | 5 | mM |
| Alamethicin | 5 | mg/mL | 2 | μL | 25 | μg/mL |

### Experimental Workflow:

The following procedures were conducted in parallel under two distinct metabolic conditions:

### a) Cofactor-Supplemented System (NADPH and UDPGA):

To the master mixture, 10 μL of liver microsomes (20 mg/mL), 40 μL of β-nicotinamide adenine dinucleotide phosphate (NADPH, 10 mM), and 40 μL of uridine 5′-diphosphoglucuronic acid (UDPGA, 20 mM) were added, yielding final concentrations of 0.5 mg/mL microsomal protein, 1 mM NADPH, and 2 mM UDPGA.

### b) Cofactor-Depleted System:

To the master mixture, 10 μL of liver microsomes (20 mg/mL) and 80 μL of ultrapure water were introduced, achieving a final microsomal protein concentration of 0.5 mg/mL.

For both systems, 398 μL of the master mixture (with or without cofactors) was dispensed into glass tubes and equilibrated in a 37° C. shaking water bath for 10 min. Reactions were initiated by adding 2 μL of a 200 μM β-amino acid-containing polypeptide or control polypeptide solution (final concentration: 1 μM) to each tube. Aliquots (50 μL) of the reaction mixture were transferred to fresh microtube plates at timed intervals (0, 15, 30, 45, and 60 min). Reactions were terminated by adding 1 volume of 1M HCl and 4 volumes of ice-cold acetonitrile containing internal standards (3% formic acid, 200 nM labetalol, and 2 μM tolfenamic acid). Samples were centrifuged (3220 g, 40 min, 4° C.), and 100 μL of the supernatant was diluted with 100 μL of ultrapure water for LC-MS/MS analysis.

### Data Analysis:

Peak areas were quantified from extracted ion chromatograms. The natural logarithm of the residual peptide percentage was plotted against incubation time to generate a linear regression curve, from which the elimination rate constant (k) was derived as the negative slope. The in vitro half-life (t½) and intrinsic clearance (CLint) were calculated as follows:

$${t}_{1 / 2} = \frac{0.693}{k} and {CL}_{int} = k \times ( \frac{Incubation volume ( μ L )}{Microsomal protein amount ( mg )} )$$

Results from triplicate determinations are summarized in Table 6.

TABLE 6
Plasma Stability and Microsomal Stability of Polypeptide

| | | | Microsomal stability | | | |
|---|---|---|---|---|---|---|
| | | | | In vitro | | In vitro |
| | | | | clearance | | clearance |
| | Plasma stability | | In vitro | rate | | rate |
| | t_{1/2} (min, | t_{1/2} (min, | t_{1/2} (min, | (μL/min/mg | t_{1/2} (min, | (μL/min/mg |
| Test article | human) | mouse) | human) | protein) | mouse) | protein) |
| glucagon | >511.69 | 95.34 | 53.37 | 25.97 | 45.61 | 30.38 |
| P001 | >511.69 | 93.59 | 53.35 | 25.98 | 53.57 | 25.87 |
| P002 | >511.69 | 100.08 | 71.90 | 19.28 | 90.58 | 15.30 |
| P003 | >511.69 | 88.70 | 70.15 | 19.76 | 79.81 | 17.37 |
| P004 | >511.69 | 80.64 | 73.17 | 18.94 | 86.54 | 16.02 |
| P005 | 490.91 | 88.46 | 68.69 | 20.18 | 85.49 | 16.21 |
| P006 | 358.43 | 82.40 | 64.73 | 21.41 | 59.63 | 23.24 |
| P007 | 310.21 | 195.21 | 78.49 | 17.66 | 59.14 | 23.44 |
| P008 | >511.69 | >511.69 | >256 | <5.42 | >256 | <5.42 |
| P009 | >511.69 | >511.69 | >256 | <5.42 | >256 | <5.42 |
| P013 | 460.45 | 366.93 | 142.27 | 9.74 | 144.80 | 9.57 |
| P015 | >511.69 | >511.69 | 201.97 | 6.86 | 160.34 | 8.64 |
| P016 | >511.69 | >511.69 | >256 | <5.42 | 249.81 | 5.55 |
| P017 | >511.69 | >511.69 | 114.25 | 12.13 | 117.17 | 11.83 |
| P018 | >511.69 | 117.57 (146.48) | >256 | <5.42 | 165.56 | 8.37 |
| P019 | >511.69 | 110.17 | >256 | <5.42 | 115.96 | 11.95 |
| Dasiglucagon | >511.69 | >511.69 | | | | |

### Data Summary:

As delineated in Table 6, the β-amino acid-containing polypeptides disclosed herein demonstrate significantly prolonged in vitro half-lives relative to glucagon under identical metabolic conditions, reflecting their enhanced metabolic stability.

### Disclaimer Clause:

The foregoing description of specific embodiments is provided for illustrative purposes and does not constrain the scope of the present invention. Practitioners skilled in the art may implement modifications, adaptations, or derivative applications of the disclosed compositions and methods, provided such alterations remain consistent with the inventive principles articulated herein. All such variations are expressly encompassed within the scope defined by the appended claims.

1. A glucagon analogue or a pharmaceutically acceptable salt thereof, comprising an amino acid sequence derived from SEQ ID NO. 1, wherein one or more α-amino acid residues at positions 2, 3, 5, 10, 13, 16, 17, 21, or 24 are substituted with β-amino acid counterparts.

2. The glucagon analogue or pharmaceutically acceptable salt thereof according to claim 1, wherein the β-amino acid is represented by Formula (I): 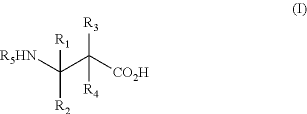
In Formula (I):
R1, R2, R3, R4, and R5 are independently selected from hydrogen or substituent groups, which may be identical or distinct; and/or
Any two of R1, R2, R3, R4, or R5, together with the α-carbon (C2) and/or β-carbon (C3) to which they are bonded, may form a cyclic structure.

3. The glucagon analogue or pharmaceutically acceptable salt thereof according to claim 1, wherein the β-amino acid is represented by Formula (II): 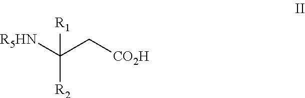
In Formula (II):
R1, R2, and R5 are independently selected from hydrogen or substituent groups, which may be identical or distinct; and/or
Any two of R1, R2, or R5, together with the β-carbon (C3) to which they are bonded, may form a cyclic structure;
preferably, R5 is hydrogen (H); more preferably, at least one of R1 or R2 is hydrogen (H).

4. The glucagon analogue or pharmaceutically acceptable salt thereof according to claim 1, wherein the β-amino acid retains the side chain (R-group) of the corresponding α-amino acid in SEQ ID NO. 1 and exists in the amino acid sequence as shown below: 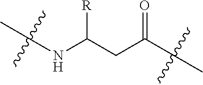
preferably, the substitution of α-amino acids with β-amino acids occurs at one or more of positions 2, 3, 5, 10, 13, 16, 17, 21, or 24 relative to SEQ ID NO. 1.

5. The glucagon analogue or pharmaceutically acceptable salt thereof according to claim 1,
wherein the analogue is a polypeptide comprising β-amino acids;
preferably, the polypeptide contains β-amino acids at one or two positions selected from 2, 3, 5, 10, 13, 16, 17, 21, or 24 of SEQ ID NO. 1;
further preferably, the substitutions comprise:
(1) βY at position 10;
(2) βS at position 16;
(3) βQ at position 24;
(4) BI) at position 21;
(5) βT at position 5;
(6) βY at position 13;
(7) βR at position 17;
(8) βS at position 2;
(9) βS at positions 2 and 16,
(10) βQ at positions 3 and 24;
(11) βQ at position 3;
(12) βS at position 16 and βQ at position 24; or
(13) βQ at position 3 and βS at position 16.

6. The glucagon analogue or pharmaceutically acceptable salt thereof according to claim 1, further comprising amino acid substitutions at one or more positions selected from 7, 10, 17, 18, 20, 21, 24, 27, 28, or 29 relative to SEQ ID NO. 1;
preferably, substitutions occur at 4-10 of these positions, selected from: T7I, Y10K, R17A, R18A, Q20A, Q20E, D21E, Q24K, Q24A, M27E, M27L, N28S, N28D, or T29E;
more preferably, the substitutions comprise:
(1) R17A; Q20B; D21E; Q24K; M27E; N28S;
(2) T7I; Y10K; R17A; Q20E; D21E; Q24K; M27E; N28S;
(3) T7I; Y10K; R18A; N28D;
(4) Q20A; M27L; N28D; T29E; or
(5) Q20A; Q24A, M27L; N28D; T29E.

7. The glucagon analogue or pharmaceutically acceptable salt thereof according to claim 1, further comprising unnatural amino acids at one or more positions selected from 2, 3, or 16 relative to SEQ ID NO. 1;
preferably, the unnatural amino acid is acetylated 2,4-diaminobutyric acid (DAB (Ac) or α-aminoisobutyric acid (Aib);
more preferably, substitutions include:
(1) Aib at position 16;
(2) Aib at position 2;
(3) DAB (Ac) at position 3; or
(4) Aib at positions 2 and 16.

8. The glucagon analogue or pharmaceutically acceptable salt thereof according to claim 1, comprising an amino acid sequence selected from SEQ ID Nos. 5-23.

9. The glucagon analogue or pharmaceutically acceptable salt thereof according to claim 1, wherein the salt is an inorganic or organic acid salt, or an inorganic or organic base salt.

10. A pharmaceutical composition comprising the glucagon analogue or pharmaceutically acceptable salt thereof according to claim 1, and optionally a pharmaceutically acceptable carrier, adjuvant, or excipient.

11. The pharmaceutical composition according to claim 10, further comprising an additional therapeutic agent;
preferably, the additional therapeutic agent is an agonist of GCGR, GLP1R, and/or GIPR.

12. A method for treating or preventing a disorder related to insulin secretion, glucagon secretion, or appetite regulation, comprising administering to a subject in need thereof a therapeutically or prophylactically effective amount of the glucagon analogue or pharmaceutically acceptable salt thereof, or the pharmaceutical composition of claim 10.

13. The method according to claim 12, wherein the disease is type 1 diabetes, type 2 diabetes, hyperglycemia, hypoglycemia, glucagon deficiency, obesity, overweight, or bulimia.
